(19) United States
(12) Patent Application Publication
(10) Pub. No.: US 2007/0090271 A1
(43) Pub. Date: Apr. 26, 2007

(54) LIGHT AMOUNT CONTROL DEVICE FOR ENDOSCOPE

(75) Inventor: Shotaro Kobayashi, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/551,491

(22) Filed: Oct. 20, 2006

(30) Foreign Application Priority Data
Oct. 21, 2005 (JP) P2005-307257

(51) Int. Cl. G01J 1/32; A61B 1/00

(52) U.S. Cl. 250/205; 600/104

(57) ABSTRACT

A light amount control device is provided in an endoscope processor. To the endoscope processor, a plurality of scopes that each has a light guide to transmit illuminating light can be selectively and detachably connected. The light amount control device includes a light source and a light amount controller. The light source emits the illuminating light. The light amount controller controls the amount of the illuminating light entering the light guide. The light amount controller controls the amount of the illuminating light, according to the light transmission characteristics of the light guide in the scope, based on scope identification information for identifying the scope that is connected to the endoscope processor.

### BACKGROUND OF THE INVENTION

1. Field of the Invention

The present invention relates to an endoscope that can control the amount of light.

2. Description of the Related Art

An endoscope device generally has a processor including a light source for illuminating a body cavity to be observed and other components, and a processor that has a light guide for transmission of illuminating light from the light source and other components, and is detachably connected to the processor. The illuminating light from the light source is transmitted by the light guide, and is emitted from the end of the scope to a subject.

Usually, a plurality of scopes, where various kinds of light guides having different quantities or diameters of fibers are provided, are connected to a processor and are used. An endoscope where a cut mask is provided at the light-entering end of a light guide, to control the generation of heat at the end of the scope, is known. The reason for such a cut mask is that when a scope including a light guide with a wide diameter is used, the amount of illuminating light emitted from the end of the scope may be excessive.

If a cut mask for reducing the amount of illuminating light entering a light guide is provided, the structure of the endoscope becomes complicated. Further, in such a case, because the diameter of the cut mask is not adjustable, adjusting the amount of illuminating light entering a light guide to be suitable according to the diameter of the light guide is impossible. In a case where a plurality of cut masks are provided, and one of them is selected for adjusting the amount of illuminating light entering a light guide, selecting a cut mask having a desirable diameter is not always possible; therefore, optimized adjustment may be impossible. In addition, providing a plurality of cut masks necessitates a more complicated structure for the endoscope.

### SUMMARY OF THE INVENTION

Therefore, an objective of the present invention is to provide an endoscope where the amount of illuminating light can be controlled according to a light guide used, and where the structure thereof is simple.

A first light amount control device, according to the present invention, is provided in an endoscope processor. To the endoscope processor, a plurality of scopes that each has a light guide to transmit illuminating light can be selectively and detachably connected. The first light amount control device includes a light source and a light amount controller. The light source emits the illuminating light. The light amount controller controls the amount of the illuminating light entering the light guide. The light amount controller controls the amount of the illuminating light according to the light transmission characteristics of the light guide in the scope, based on scope identification information for identifying the scope that is connected to the endoscope processor.

The first light amount control device may further include a determination processor that determines whether the amount of the illuminating light should be lowered or not, based on the scope identification information. The light amount controller may lower the amount of the illuminating light when the determination processor determines that the amount of the illuminating light should be lowered.

The first light amount control device may further include a data memory in which light amount control data is stored, the data memory being for controlling the amount of the illuminating light, and being set for each of the scopes. The light amount controller may control the amount of the illuminating light based on the light amount control data of the scope, the scope being identified based on the scope identification information.

The scope identification information can be entered into the first light amount control device externally.

The light amount controller may include an aperture and an aperture controller that controls the position of the aperture. The aperture controller may control the position of the aperture according to a command of a user.

The first light amount control device may further include a photosensor and a mode selector. The photosensor may receive reflected light of the illuminating light reflected on a subject and detect the amount of the reflected light. The mode selector may select either an automatic optical control mode, where the aperture controller controls the position of the aperture according to the amount of the reflected light, or a manual optical control mode, where the aperture controller controls the position of the aperture according to a command of a user.

In the first light amount control device, the narrower the diameter of the light guide, the lower the amount of the illuminating light may be controlled by the light amount controller.

A second light amount control device, according to the present invention, is provided in an endoscope processor, to which a plurality of scopes that each has a light guide to transmit illuminating light can be selectively and detachably connected. The light amount control device includes a light source, a scope identifier, an operation member, and a light amount controller. The light source emits the illuminating light. The scope identifier identifies the scope that is connected to the endoscope processor. The light amount controller controls the amount of the illuminating light entering the light guide. The light amount controller controls the amount of the illuminating light in stages when the operation member is operated, and the difference between the stages is according to the type of the scope that is identified.

An endoscope according to the present invention includes a plurality of scopes and an endoscope processor. Each of the scopes has a light guide to transmit illuminating light. To the endoscope processor, the scopes can be selectively and detachably connected. The endoscope processor includes a light amount control device. The light amount control device includes a light source and a light amount controller. The light source emits the illuminating light. The light amount controller controls the amount of the illuminating light according to the light transmission characteristics of the light guide in the scope, based on scope identification information for identifying the scope that is connected to the endoscope processor.

### BRIEF DESCRIPTION OF THE DRAWINGS

The present invention will be better understood from the description of the preferred embodiment of the invention set forth below, together with the accompanying drawings, in which:

FIG. 1 is a block diagram of an electronic endoscope of the embodiment;

FIG. 2 is a block diagram of a comparative example of an electronic endoscope;

FIG. 3 is a side view approximately representing a lighting unit and an aperture;

FIG. 4 is a perspective view approximately representing a lighting unit and an aperture;

FIG. 5 is a front view approximately representing a lighting unit and an aperture seen from a light guide side;

FIG. 6 is a view representing examples of voltages in accordance with light amount levels set in a manual optical control mode, and corresponding positions of the aperture; and

FIG. 7 is a view representing examples of voltages in accordance with light amount levels lower than those represented in FIG. 6, and corresponding positions of the aperture.

### DESCRIPTION OF THE PREFERRED EMBODIMENT

Hereinafter, the preferred embodiment of the present invention is described with reference to the attached drawings.

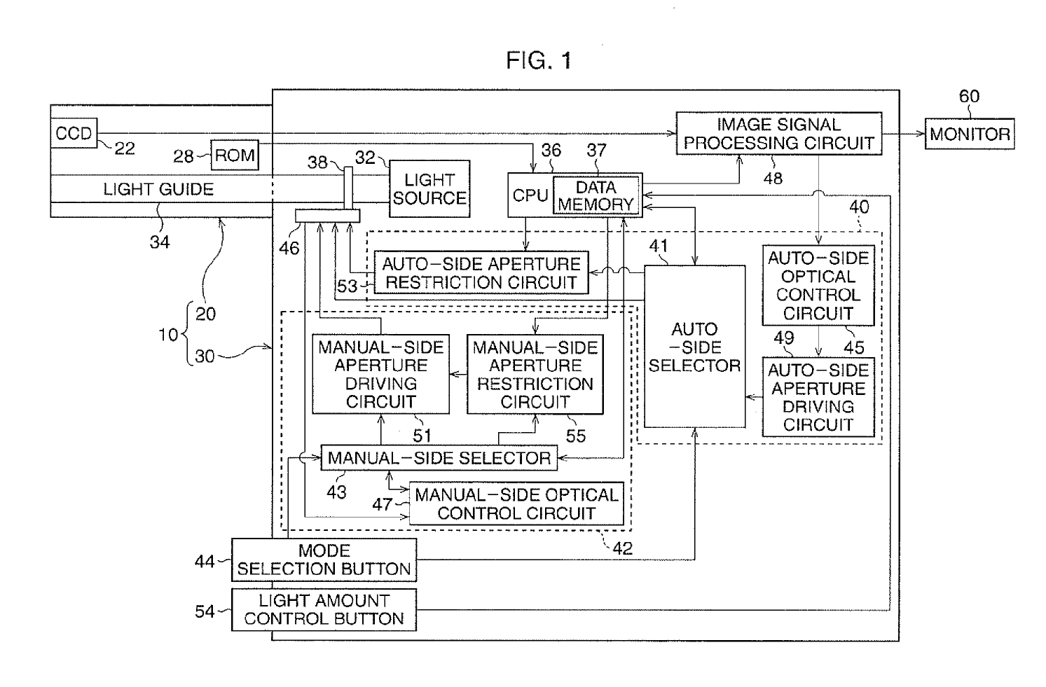

As shown in FIG. 1, an electronic endoscope 10 includes a scope 20 and a processor 30. The scope 20 is used for observing and photographing inside a body cavity of a subject person. The processor 30 provides illuminating light for the scope 20, and processes image signals transferred from the scope 20. The scope 20 is selectively and detachably connected to the processor 30. To the processor 30, a plurality of different scopes, including the scope 20, can be connected, and one of the scopes selected by a user is connected to the processor 30 and used. Further, a monitor 60 is connected to the processor 30.

In the processor 30, a CPU 36 for controlling the entirety of the processor 30, and a lighting unit 32, which emits illuminating light for illuminating a subject, are provided. When electronic power is supplied under the control of the CPU 36, the lighting unit 32 emits the illuminating light. The emitted illuminating light enters the light guide 34 after passing through a collective lens (not shown) and an aperture 38 that is driven by an aperture driving motor 46. The light guide 34 transmits the entered illuminating light to the end of the scope 20, then the illuminating light passing through the light guide 34 is emitted to a subject.

The reflected light of the illuminating light reflected on a subject, passes through an objective lens (not shown) and a color filter (not shown), then reaches a CCD 22. As a result of this, a charge for generating image signals of a subject image in accordance with colors of reflected light which has passed through the color filter, is generated by photoelectric conversion. Then, the charge is accumulated on a light-receiving surface of the CCD 22. Image signals generated by charge accumulated on the light-receiving surface of the CCD 22 are read successively at intervals of a predetermined period.

The read image signals are amplified and converted from analog image signals to digital image signals. Further, various processes, such as white balance adjustment, are carried out on the digital image signals, then luminance signals and color-difference signals are generated. The luminance signals and color-difference signals are transmitted to an image signal processing circuit 48 in the processor 30, and are then converted to image signals, including NTSC signals. The image signals are output to the monitor 60. As a result, a subject image is displayed on the monitor 60.

Note that in the scope 20, a scope controller (not shown) for controlling the entirety of the scope 20, a ROM 28 in which data relating to the characteristics of the scope 20 and processing of signals are pre-stored, and other elements are provided. Data stored in the ROM 28 are read by the scope controller, then are used.

In the ROM 28, scope identification information for identifying the scope that is connected to the processor 30 is also stored. The scope identification information, for example, can represent a model number for each of the scopes connectable to the processor 30, and is read by the CPU 36 in the processor 30. Based on the read scope identification information, it is identified, by the CPU 36 that has read the scope identification information that the scope connected to the processor 30 at the time is the scope 20. Further, the light guide currently used in the scope 20 is identified to be the light guide 34, based on the scope identification information. In the processor 30, the amount of light entering the light guide 34 is adjusted in accordance with the type of the light guide that is in use, as explained below.

In the processor 30, either an automatic optical control mode where the amount of the light entering the light guide 34 is automatically adjusted, or an manual optical control mode where the amount of the light entering the light guide 34 is adjusted in accordance with a command of a user, can be selected. That is, when an optical mode selection button 44 provided on a front panel (not shown) on the surface of the processor 30, is depressed, the manual optical control mode is set. On the other hand, when the optical mode selection button 44 is not depressed after the electronic endoscope 10 starts, or when the optical mode selection button 44 is depressed again after the manual optical control mode is set, the automatic optical control mode is set.

A light amount control button 54 is provided close to the mode selection button 44. While the manual optical control mode is set, the amount of light entering the light guide 34 can be adjusted in stages to a desired level, by depression of the light amount control button 54.

In the processor 30, an automatic optical control unit 40 for controlling the amount of light entering the light guide 34 in the automatic optical control mode, and a manual-side optical control unit 42 for controlling the amount of light in the manual optical control circuit mode, are provided. In the automatic optical control unit 40 and the manual-side optical control unit 42, an auto-side selector 41 and a manual-side selector 43 are provided respectively. When the auto-side and manual-side selectors 41 and 43 receive signals generated by the depression of the optical mode selection button 44, signals corresponding to the received signals are transmitted to the CPU 36. As a result, the automatic optical control mode and the manual optical control mode are reversed, and the selected mode is set.

In the CPU 36, a data memory 37 is provided. In the data memory 37, light amount control data for all the scopes, including the scope 20 that can be used with the processor 30, are stored as table data. The light amount control data is used for controlling the amount of the illuminating light; and include a diameter-setting voltage “Vs” that is a voltage for the aperture driving motor 46 to set the maximum opening diameter of the aperture 38, and a correction coefficient “α”, as explained below.

Note that the light amount control data is stored as a table to enable suitable control of the light amount, based on the light transmission characteristics of each of the light guides in the scopes; that is, the number of fibers, their diameters, their lengths, their materials, or other such characteristics, such as the output angles of the scopes.

When the scope 20 is connected to the processor 30, the CPU 36 reads the scope identification information from the ROM 28, then, the scope connected to the processor 30 and in use is identified to be the scope 20. Further, based on the read identification information, the CPU 36 reads the light amount control data of the light guide 34 in the scope 20 that is identified to be in use, from the data memory 37. The CPU 36 determines whether the amount of the illuminating light entering the light guide 34 should be lowered or not, based on the light amount control data of the light guide 34, as explained below. This control of the amount of the illuminating light entering the light guide 34 using the light amount control data is explained below, for each of the optical control modes.

When the automatic optical control mode is set, luminance signals are transmitted from the image signal processing circuit 48 to an auto-side optical control circuit 45 provided in the automatic optical control unit 40, under the control of the CPU 36. In the auto-side optical control circuit 45, the difference between the level of the received luminance signals that corresponds to the amount of the reflected light of the illuminating light detected by the CCD 22, and the predetermined suitable luminance level, is calculated. Then, based on the calculated difference between the luminance levels, an auto-side aperture driving circuit 49 applies operating voltage that is required for moving the aperture 38 to a predetermined position to the auto-side selector 41, for the purpose of controlling the amount of the illuminating light entering the light guide 34.

Then, the CPU 36 determines whether the amount of the illuminating light entering the light guide 34 would be excessive or not if the aperture 3B were moved, based on the voltage applied from the auto-side aperture driving circuit 49 to the auto-side selector 41. This determination is carried out based on the light amount control data (the diameter-setting voltage “Vs”, represented by the light amount control data, as explained below) previously obtained.

If it is determined that the amount of the illuminating light entering the light guide 34 would be suitable if the aperture 38 were moved based on the applied operating voltage, then further control of the amount of light to the light guide 34 is not required; that is, decreasing the light amount is not required, (i.e., it is determined that the diameter-setting voltage “Vs”, represented by the obtained light amount control data, is equal to that of predetermined standard data, so that decreasing the light mount is not required, as explained below), and the applied operating voltage is further applied to the aperture driving motor 46 in the current state. As a result, the aperture 38 is moved by the aperture driving motor 46 (to which the predetermined amount of operating voltage is applied) to the position calculated by the auto-side optical control circuit 45, and the light amount entering the light guide is controlled. That is, the position of the aperture 38 and the light amount are set corresponding to the operating voltage.

On the other hand, if it is determined by the CPU 36 that the amount of illuminating light entering the light guide 34 would be in excess over the suitable amount if the aperture 38 were moved based on the applied operating voltage, then decreasing the amount of light to the light guide 34 becomes required (i.e., it is determined that the diameter-setting voltage “Vs”, represented by the obtained light amount control data, is lower than that of predetermined standard data, so that decreasing the light mount would be required, as explained below), and signals representing this fact are transmitted from the CPU 36 to the auto-side selector 41. Further, the CPU 36 transmits other signals representing how much the light amount to the light guide 34 should be decreased (that is, how much the aperture 38 should be moved to the closing side (i.e., moved to a position where the optical path of the illuminating light is closed)) to an auto-side aperture restriction circuit 53.

Note that, for example, when a scope that includes a light guide having a narrow diameter is connected to the processor 30, the amount of the illuminating light is decreased, because it is determined that the amount of illuminating light entering the light guide 34 would be in excess of the suitable amount. At that time, in principle, the narrower the diameter of the light guide included in the scope, the lower the amount of the illuminating light controlled.

In this case, unlike the above-explained case where the light amount is suitable, the operating voltage applied from the auto-side aperture driving circuit 49 to the auto-side selector 41 is further applied to the aperture driving motor 46 via the auto-side aperture restriction circuit 53. Then, the auto-side aperture restriction circuit 53 corrects the operating voltage from the auto-side aperture driving circuit 49, via the auto-side selector 41, based on the signals transmitted from the CPU 36 for this correction, so that the corrected voltage, not the original operating voltage, is applied to the aperture driving motor 46. As a result, compared to the case in which the CPU 36 determines that decreasing the amount of the illuminating light is not required, the aperture 38 is moved to a position where the optical path of the illuminating light is more blocked, thus the amount of the illuminating light entering the light guide 34 is controlled.

Note that the standard data represent a standard voltage that is a reference value for determining whether light amount control is carried out or not. The standard voltage is previously set according to the combination of a processor and one of the scopes that are used with the processor, based on the diameter of the light guide included in the scope and other factors. Then, if the above scope is connected to the processor, the standard voltage is, in principle, equal to the diameter-setting voltage “Vs”, so that the amount of the illuminating light entering the light guide is not controlled. On the other hand, if one of the other scopes is connected to the processor, the standard voltage is not equal to the diameter-setting voltage “Vs”, so that the amount of the illuminating light is controlled.

On the other hand, when the manual optical control mode is set, signals representing the light amount level commanded by a user by depression of the light amount control button 54 are transmitted to the CPU 36 via the manual-side selector 43. The CPU 36 then determines whether the amount of the illuminating light entering the light guide 34 should be decreased or not, based on the optical control data of the light guide 34 read from the data memory 37, similarly to when the automatic optical control mode is set.

If it is determined by the CPU 36 that the amount of the illuminating light should not be decreased (i.e., if it is determined that the diameter-setting voltage “Vs” and the correction coefficient “α” represented by the obtained light amount control data are equal to those of predetermined standard data, so that decreasing the light mount is not required, as explained below), the manual-side selector 43 is controlled, so that the position control signals which are for controlling the position of the aperture 38 and which are output from a manual-side optical control circuit 47, are transmitted to a manual-side aperture driving circuit 51 directly, not via a manual-side aperture restriction circuit 55. Here, the position control signals are signals for driving the aperture 38 to be moved to a position where the illuminating light, whose amount corresponds to the commanded light amount level, enters the light guide 34.

The aperture driving motor 46 is a servomotor, where the position of the rotation axis varies in accordance with the amount of operating voltage. The position of the rotation axis corresponds to the position of the aperture 38, and position data representing the position of the aperture 38 are transmitted to the manual-side optical control circuit 47 as aperture position data. The manual-side optical control circuit 47 determines whether the actual position of the aperture 38 is suitable position or not, based on the position control signals and the aperture position data. Then, the manual-side optical control circuit 47 controls the standard voltage to be provided to the aperture driving motor 46 according to need.

On the other hand, if it is determined by the CPU 36 that the amount of the illuminating light should be decreased (i.e., if it is determined that the diameter-setting voltage “Vs” and the correction coefficient “a” represented by the obtained light amount control data are different from those of predetermined standard data, so that decreasing the light amount is required, as explained below), the manual-side selector 43 is switched to the side of the manual-side aperture restriction circuit 55, so that the position control signals from the manual-side optical control circuit 47 are output to the manual-side aperture restriction circuit 55. Then, the corrected position control signals, which are corrected in the manual-side aperture restriction circuit 55, are input to the aperture driving motor 46 via the manual-side aperture driving circuit 51, as explained below.

That is, first, the CPU 36 transmits the correction coefficient “α” in the obtained light amount control data to the manual-side aperture restriction circuit 55. In the manual-side aperture restriction circuit 55, the received position control signals are multiplied by the correction coefficient “α” to be corrected position control signals that are suitable for the light guide 34 in the scope 20 that is the scope connected to the processor 30, and then the corrected position control signals are transmitted to the manual-side aperture driving circuit 51. The standard voltage to be applied to the aperture driving motor 46 is adjusted, based on the corrected position control signals in the manual-side aperture driving circuit 51.

As a result, the aperture 38 is moved to a suitable position, and a lower amount of the illuminating light than the light amount corresponding to the light amount level commanded by a user enters the light guide 34.

Note that the aperture driving motor 46 is adjusted, at the starting time thereof, by the manual-side optical control circuit 47, so that the opening angle of the aperture 38 is maximum when the maximum standard voltage is applied to the aperture driving motor 46, whereas the opening angle of the aperture 38 is minimum when the minimum standard voltage is applied to the aperture driving motor 46 (as explained below). Due to this adjustment, the manual-side optical control circuit 47 prevents a negative impact on the light amount control due to individual differences in the aperture driving motors. The reason is that the maximum and the minimum opening angles of the aperture 38 can be steady, even if a small error is included in the applied voltage due to individual difference in the aperture driving motors.

When the manual optical control mode is set, the position of the aperture control circuit is feedback-controlled by the manual-side optical control circuit 47. That is, the actual position of the aperture driving motor 46 is detected, and the detected actual position is compared with the position corresponding to the light amount level set by a user, by the manual-side optical control circuit 47. If there is a difference between these positions, signals corresponding to the difference are output to the manual-side aperture driving circuit 51, so that the aperture driving motor 46 is moved to the suitable position. Further, signals representing the position of the aperture driving motor 46 after being moved are transmitted to the manual-side optical control circuit 47. By repeating the feedback control, the aperture 38 is moved to the suitable position corresponding to the light amount level selected by a user, so that the amount of the illuminating light entering the light guide 34 is accurately controlled.

In the electronic endoscope 10 of this embodiment, where the light guide in use is identified and the amount of the illuminating light entering the light guide is adjustable in accordance with the characteristics of the light guide, as explained above, problems such as excessively high temperature near the light-emitting end of the light guide 34 (that is, the end of the scope 20), which may be caused by an excessive amount of illuminating light entering the light guide 34, can be prevented.

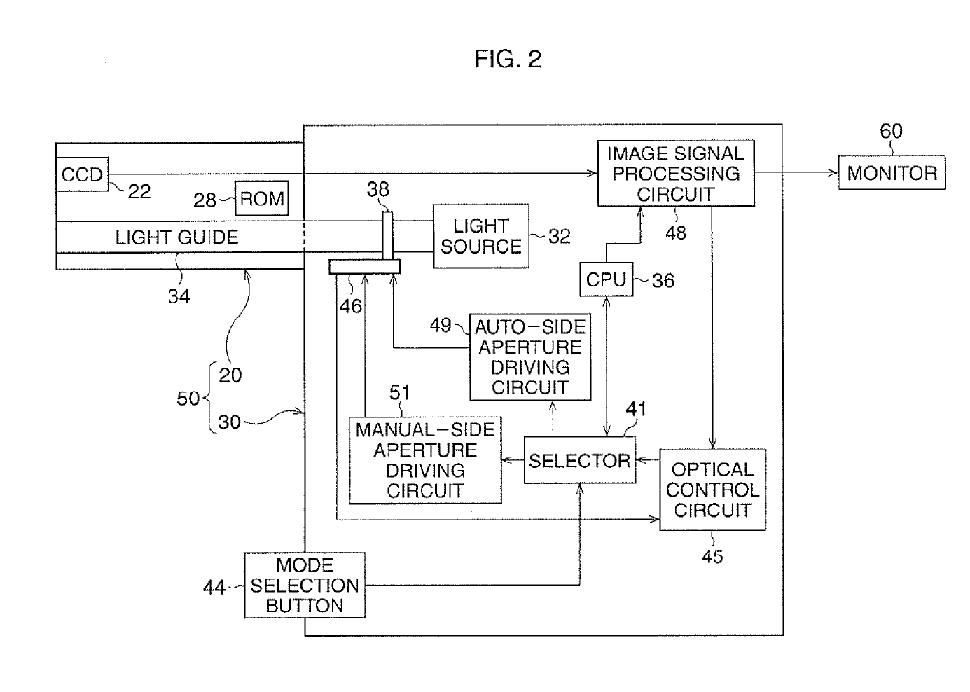

On the other hand, in an electronic endoscope 50 of the comparative example (see FIG. 2), some scopes can not be used with the processor 30, because the amount of the illuminating light is not suitably adjusted, and problems such as the aforementioned one of increasing temperature may be caused. The reason for such problems is that, in the electronic endoscope 50 of the comparative example, the light guide in use can not be identified, and a data memory, such as the data memory 37 for storing the light amount control data, is not provided.

Note that in some cases, setting either the automatic optical control mode or the manual optical control mode is preferable. For example, when a scope, to which an add-on camera can be connected, is connected to the processor 30, the amount of the illuminating light should be controlled by the manual-side optical control unit 42, because image signals from a CCD are not transmitted to the processor 30. In such a case, setting the optical control mode selectively, based on factors other than the optical characteristics of the light guide 34, can be carried out by adding the required information to the optical control data.

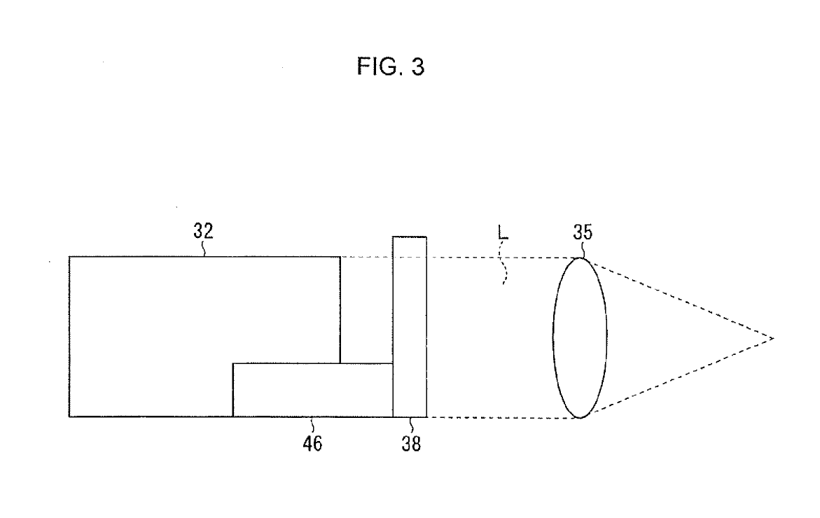

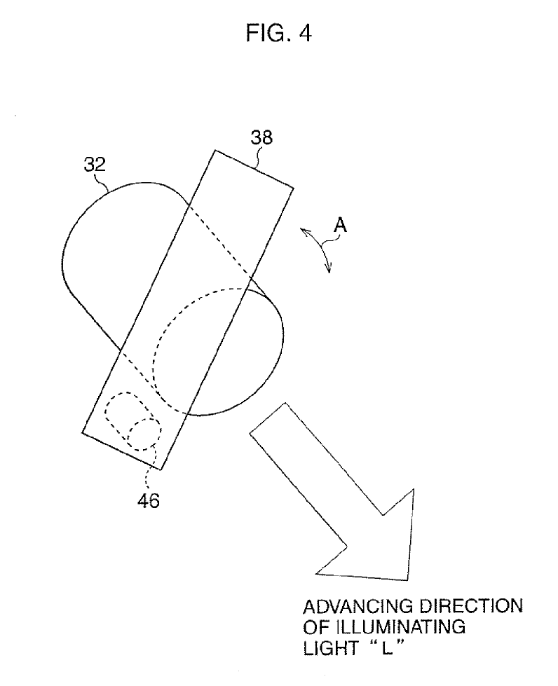

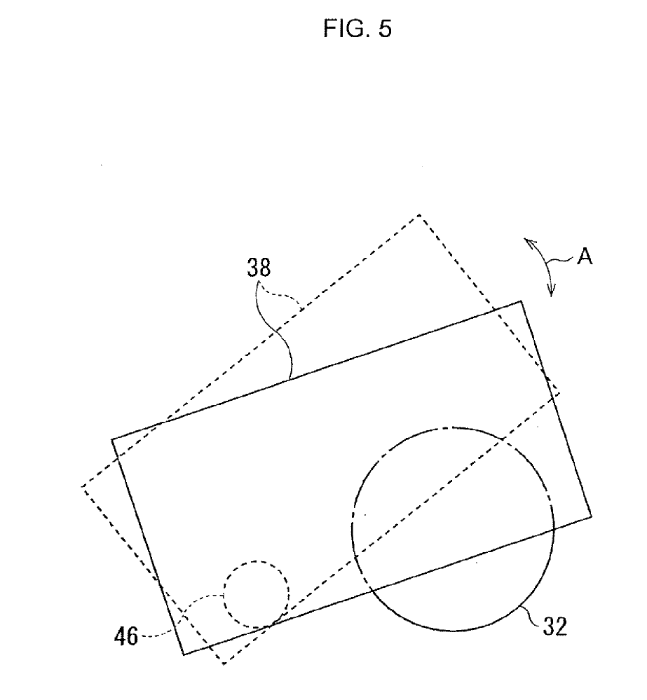

The aperture 38 has a feather shape, and is attached to the aperture driving motor 46 (see FIGS. 3 and 4). The illuminating light “L”, passing through the aperture 38, enters the light guide 34 (see FIG. 1) after being collected by the collective lens 35. The aperture 38 rotates in a perpendicular direction to the optical path of the illuminating light “L”, as the arrow A represents. The aperture driving motor 46 that is controlled by the automatic optical control unit 40 or the manual-side optical control unit 42 drives the aperture 38 to a predetermined rotational position (see FIGS. 4 and 5).

Note that FIGS. 3 to 5 represent the composition and arrangement of the lighting unit 32, the aperture 3B, and other components approximately, and the actual composition and arrangement of the lighting unit 32, the aperture 38, and other components are not limited to those represented in these figures. For example, the aperture 38 may not be designed to shut out preferentially the end of the optical path of the illuminating light, as represented in FIGS. 4 and 5, but may be designed to shut out both the center area and the end of the optical path almost equally.

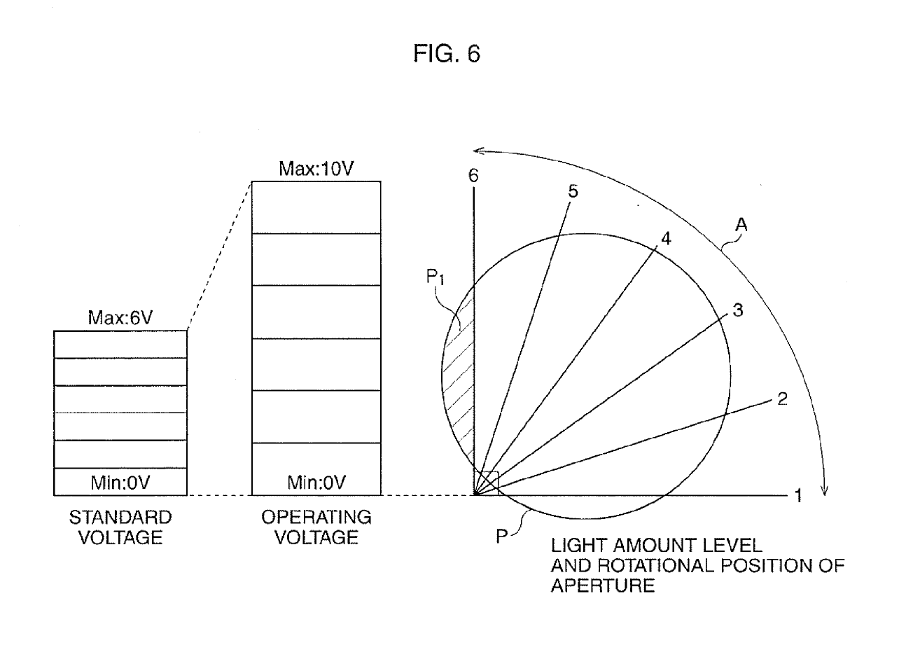

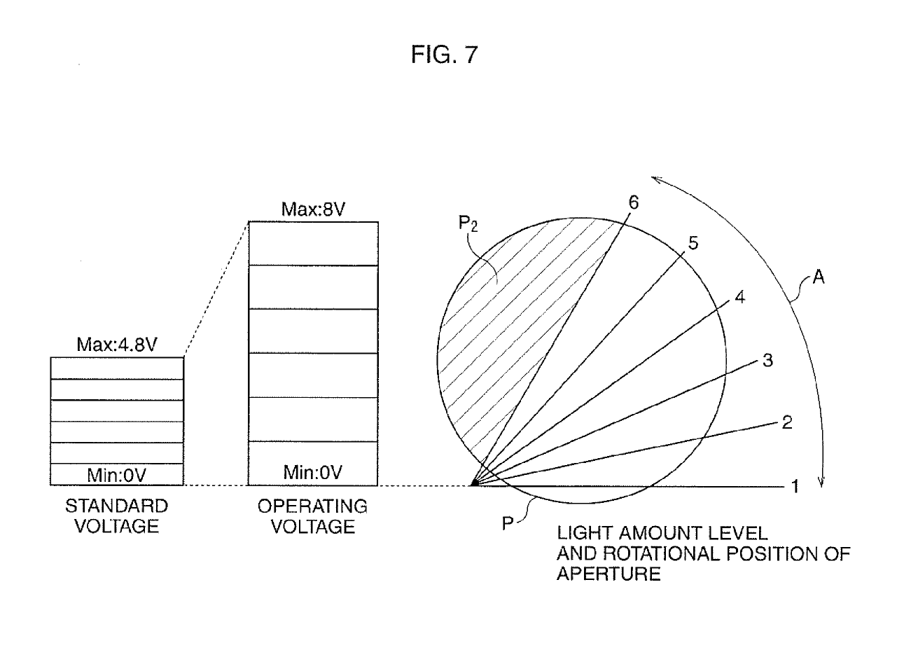

In the manual optical control mode, the light amount of the illuminating light used for observing and illuminating a subject, for example, is set in six levels (see FIGS. 6 and 7). When it is determined by the CPU 36 that decreasing the amount of the light entering the light guide 34 is not required, one of the standard voltages corresponding to each of the light amount levels represented in FIG. 6. is applied to the aperture driving motor 46.

On the other hand, as explained above, when it is determined by the CPU 36 that decreasing the amount of the light entering the light guide 34 is required, the manual-side aperture driving circuit 51 lowers the standard voltages to be applied to the aperture driving motor 46, based on the signals transmitted from the manual-side aperture restriction circuit 55. Namely, for example, the standard voltage to be applied to the aperture driving motor 46 is 6.0 (V) at the maximum, when the standard voltage is not controlled, and the maximum standard voltage is lowered to 4.8 (V) by multiplying the standard voltage by the correction coefficient “α” of 0.8 (see FIG. 7). Similarly to with the standard voltages, operating voltages corresponding to the standard voltages are lowered, for example, from 10 (V) to 8 (V) at maximum.

As a result, the differential angle of the rotational positions of the aperture 38, between the rotational position corresponding to the lowest light amount level (that is, light amount level 1, where the amount of the illuminating light “L” passing through the aperture 38 is the lowest), and the other rotational position corresponding to the highest light amount level (that is, light amount level 6, where the amount of the illuminating light “L” passing through the aperture 38 is the highest) is, for example, approximately 90 degrees (see FIG. 6). On the other hand, when the standard voltages are lowered, the differential angle is reduced to approximately 60 degrees (see FIG. 7). Note that the lines on the optical path “P” of the illuminating light “L” in FIGS. 6 and 7 represent the position of the lower end of the aperture 3B when each light amount level is set.

When light amount level 6 is set and it is determined that decreasing the light amount is not required, a first shut-out area P₁ (that is, an area shut out by the aperture 38 in the optical path “P” of the illuminating light “L”) is very small (see FIG. 6). On the other hand, when the same light amount level 6 is set and it is determined that decreasing the light amount is required, a second shut-out area P₂ (that is, an area shut out by the aperture 38 in the optical path “P” of the illuminating light “L” is relatively large compared to the first shut-out area P₁ (see FIG. 7).

As it is clear from the comparison of FIGS. 6 and 7, a shut-out area shut out by the aperture 38 is larger when it is determined that decreasing the incident light amount is required, and the standard voltages are lowered, than when it is determined that decreasing the incident light amount is not required, and the standard voltages are not lowered. This is true regardless of the set light amount level, that is, it is true not only when the light amount level 6 is set as exemplified, but also when the other light amount level is set. Note that if the standard voltages are lowered, the difference between the rotational positions of the aperture 38 in the different light amount levels is reduced, that is, the rotational amount of the aperture 38 when a different light amount level is set is reduced.

For example, if the standard voltages to be applied to the aperture driving motor 46 are adjusted by using the correction coefficient “α” (where “α”=0.8, in this example) as explained above, variational differences in the standard voltages and the operating voltages, and the difference in the rotational positions of the aperture 38 (i.e., the difference in the opening angle), are reduced to 0.8 times those if the standard values are not adjusted, when the light amount level is changed by one level. Note that the differential amount of the operating voltages between the predetermined levels is different, according to the type of the scope in use, because the light amount control data, including a correction coefficient “α”, are set for each of the scopes to be used with the processor 30 so that the value of the correction coefficient “α” is not limited to 0.8 in the example.

In the electronic endoscope 10 of this embodiment, as explained above, the scope identification information for identifying the scope 20 and the light guide 34, is stored in the ROM 28 of the scope 20, and the amount of the illuminating light entering the light guide 34 is controlled by using the light amount control data, based on the light transmission characteristics of the light guide 34 in use, so that the amount of the illuminating light is adjusted to a suitable level. Therefore, the problems of excessive focused illuminating light causing an excessively high temperature in, or breakage of the light guide 34, can be prevented.

The scope identification information may be input externally to the processor 30; that is, it may possible to enter the scope identification information by such a device as a keyboard connected to the processor 30. Further, the light amount control data may be stored in each of the scopes that are connectable to the processor 30, instead of in the ROM 28, and the light amount control data may be read by the CPU 36 each time a scope is connected to the processor 30, without identification of the connected scope and the associated light guide.

Note that light guide data that directly represent the light transmission characteristics of the light guide may be used, instead of the tabled light amount control data.

In such a case, first, it is determined whether the amount of the illuminating light should be decreased or not, based on the light guide data representing the diameter of the light guide (the number and diameter of optical fibers included in the light guide), the length of the light guide that determines the attenuation ratio thereof, and other factors. Then, when it is determined that the amount of the illuminating light should be decreased, the actual amount of the illuminating light entering the light guide is rendered lower than the amount determined suitable by the automatic optical control unit 40 or the manual-side optical control unit 42.

Note that the light amount adjustment in the processor 30 may not be limited to lowering the amount of the illuminating light passing through the aperture 38 and entering the light guide 34. For example, in a processor where a lighting unit included therein emits a relatively low amount of illuminating light, the aperture driving motor 46 may be caused to increase the amount of illuminating light passing through the aperture 38, according to the light guide that is in use.

The invention is not limited to that described in the preferred embodiment; namely, various improvements and changes may be made to the present invention without departing from the spirit and scope thereof.

The present disclosure relates to subject matter contained in Japanese Patent Application No. 2005-307257 (filed on Oct. 21, 2005) which is expressly incorporated herein, by reference, in its entirety.

1. A light amount control device provided in an endoscope processor, to which a plurality of scopes that each has a light guide to transmit illuminating light can be selectively and detachably connected, said light amount control device comprising:
a light source that emits said illuminating light; and
a light amount controller that controls the amount of said illuminating light entering said light guide, said light amount controller controlling the amount of said illuminating light according to light transmission characteristics of said light guide in said scope, based on scope identification information for identifying said scope that is connected to said endoscope processor.

2. The light amount control device according to claim 1, further comprising a determination processor that determines whether the amount of said illuminating light should be lowered or not, based on said scope identification information, wherein said light amount controller lowers the amount of said illuminating light when said determination processor determines that the amount of said illuminating light should be lowered.

3. The light amount control device according to claim 1, further comprising a data memory in which light amount control data is stored, said data memory being for controlling the amount of said illuminating light and being set for each of said scopes, wherein said light amount controller controls the amount of said illuminating light based on said light amount control data of said scope, said scope being identified based on said scope identification information.

4. The light amount control device according to claim 1, wherein said scope identification information can be entered into said light amount control device externally.

5. The light amount control device according to claim 1, wherein said light amount controller comprises an aperture and an aperture controller that controls the position of said aperture.

6. The light amount control device according to claim 5, wherein said aperture controller controls said position of said aperture, according to a command of a user.

7. The light amount control device according to claim 5, further comprising a photosensor that receives reflected light of said illuminating light reflected on a subject, and that detects the amount of said reflected light; and a mode selector that selects either an automatic optical control mode where said aperture controller controls a position of said aperture according to the amount of said reflected light, or an manual optical control mode where said aperture controller controls a position of said aperture according to a command of a user.

8. The light amount control device according to claim 1, wherein the narrower the diameter of said light guide, the lower the amount of said illuminating light controlled by said light amount controller.

9. A light amount control device provided in an endoscope processor, to which a plurality of scopes that each has a light guide to transmit illuminating light can be selectively and detachably connected, said light amount control device comprising:
a light source that emits said illuminating light;
a scope identifier that identifies said scope that is connected to said endoscope processor;
an operation member; and
a light amount controller that controls the amount of said illuminating light entering said light guide, said light amount controller controlling the amount of said illuminating light in stages when said operation member is operated, the difference between said stages being according to the type of said scope that is identified.

10. An endoscope comprising:
a plurality of scopes that each has a light guide to transmit illuminating light; and
an endoscope processor to which said plurality of scopes can be selectively and detachably connected;
said endoscope processor comprising a light amount control device, said light amount control device comprising:
a light source that emits said illuminating light; and
a light amount controller that controls the amount of said illuminating light entering said light guide, said light amount controller controlling the amount of said illuminating light according to light transmission characteristics of said light guide in said scope, based on scope identification information for identifying said scope that is connected to said endoscope processor.
